(19) United States
(12) Patent Application Publication
(10) Pub. No.: US 2020/0170623 A1
(43) Pub. Date: Jun. 4, 2020

(54) METHODS FOR USING RADIAL ENDOBRONCHIAL ULTRASOUND PROBES FOR THREE-DIMENSIONAL RECONSTRUCTION OF IMAGES AND IMPROVED TARGET LOCALIZATION

(71) Applicant: Dorian AVERBUCH, Ramat Hasharon (IL)

(72) Inventor: Dorian AVERBUCH, Ramat Hasharon (IL)

(73) Assignee: Body Vision Medical Ltd., Ramat Hasharon (IL)

(21) Appl. No.: 16/615,721

(22) PCT Filed: May 24, 2018
(86) PCT No.: PCT/IB2018/000624

§ 371 Date: Nov. 21, 2019

Related U.S. Application Data
(60) Provisional application No. 62/510,729, filed on May 24, 2017.

(51) Int. Cl. A61B 8/08; A61B 8/00; A61B 8/12; A61B 90/00; G16H 30/40; G16H 50/20; G16H 50/50

(52) U.S. Cl. CPC A61B 8/483; A61B 8/08; A61B 8/4245; A61B 8/5261; A61B 8/12; A61B 90/37; A61B 2090/3784; G16H 50/20; G16H 50/50; A61B 2090/367; A61B 2090/3762; A61B 2090/3966; G16H 30/40

(57) ABSTRACT

A method, including obtaining at least one preoperative image from an imaging modality; identifying, on the at least one preoperative image, at least one element located within an area of interest; obtaining at least one intraoperative image; highlighting the at least one element on the at least one intraoperative image; navigating a radial endobronchial ultrasound probe to the area of interest using the at least highlighted at least one element; acquiring a plurality of radial endobronchial ultrasound images; extracting a plurality of two-dimensional representations of the element, each of the plurality of two-dimensional representations of the element being extracted from a corresponding one of the plurality of radial endobronchial ultrasound images; reconstructing a three-dimensional representation of the element from the plurality of two-dimensional representations of the element; and projecting a two-dimensional projection of the three-dimensional representation of the element on at least one of the at least one intraoperative image.

### CROSS-REFERENCE TO RELATED APPLICATION

This application is an international (PCT) application relating to and claiming the benefit of commonly-owned, copending U.S. Provisional Patent Application No. 62/510,729, entitled “METHODS FOR USING RADIAL ENDOBRONCHIAL ULTRASOUND PROBES FOR THREE-DIMENSIONAL RECONSTRUCTION OF IMAGES AND IMPROVED TARGET LOCALIZATION,” filed May 24, 2017, the contents of which are incorporated by reference herein in their entirety.

### FIELD OF THE INVENTION

The present invention relates to medical imaging. More particularly, the present invention relates to methods involving the use of radial endobronchial ultrasound. More particularly, the present invention relates to methods involving the use of images obtained using radial endobronchial ultrasound imaging to provide localization of targets, such as lesions, in medical images.

### BACKGROUND

Radial endobronchial ultrasound is a medical imaging technique whereby ultrasound waves are emitted radially from a probe positioned within a bronchial passageway of a patient. The ultrasound waves are processed to produce a medical image showing a cross-section (e.g., a “slice”) of the patient's tissue around the bronchial passageway.

### SUMMARY

In an embodiment, a method includes obtaining at least one preoperative image from an imaging modality; identifying, on the at least one preoperative image, at least one element located within an area of interest; obtaining at least one intraoperative image; highlighting the at least one element on the at least one intraoperative image; navigating a radial endobronchial ultrasound probe to the area of interest using the at least highlighted at least one element; acquiring a plurality of radial endobronchial ultrasound images; extracting a plurality of two-dimensional representations of the element, each of the plurality of two-dimensional representations of the element being extracted from a corresponding one of the plurality of radial endobronchial ultrasound images; reconstructing a three-dimensional representation of the element from the plurality of two-dimensional representations of the element; and projecting a two-dimensional projection of the three-dimensional representation of the element on at least one of the at least one intraoperative image.

In an embodiment, the step of projecting the two-dimensional projection of the three-dimensional representation of the element on the at least one of the at least one intraoperative image is performed in real time.

In an embodiment, a method includes removing the radial endobronchial ultrasound probe from the area of interest; and navigating a further endobronchial tool to the area of interest. In an embodiment, a method includes performing a procedure on the element using the further endobronchial tool. In an embodiment, a method includes removing the further endobronchial tool; navigating the radial endobronchial ultrasound probe to the area of interest; acquiring a plurality of updated radial endobronchial ultrasound images; extracting a plurality of updated two-dimensional representations of the element, each of the plurality of updated two-dimensional representations of the element being extracted from a corresponding one of the plurality of updated radial endobronchial ultrasound images; and reconstructing an updated three-dimensional representation of the element from the plurality of two-dimensional representations of the element.

In an embodiment, a method includes calculating distances between a center of the radial endobronchial ultrasound probe and a plurality of boundary points on a boundary of the target; and estimating a margin size for an ablation based on a maximum one of the distances. In an embodiment, the at least one intraoperative image includes an X-ray.

In an embodiment, the three-dimensional representation of the element is used as a prior for volume reconstruction from at least one of the intraoperative images. In an embodiment, a method also includes registering the three-dimensional representation of the target to a three-dimensional computed tomography volume; and projecting the three-dimensional representation of the element from the three-dimensional computed tomography volume on at least one of the at least one intraoperative image. In an embodiment, the three-dimensional computed tomography volume is a preoperative computed tomography scan volume or a three-dimensional computed tomography volume reconstructed from the at least one intraoperative image.

In an embodiment, a method includes navigating a radial endobronchial ultrasound probe to an area of interest; acquiring a plurality of radial endobronchial ultrasound images and a plurality of intraoperative images, each of the plurality of radial endobronchial ultrasound images corresponding to one of the plurality of intraoperative images and to a different position of the ultrasound probe; extracting a radial endobronchial ultrasound probe tip position from each of the intraoperative images; generating a database of pairs of the intraoperative and endobronchial ultrasound images, each pair corresponding to a specific probe tip position and orientation in the preoperative image coordinate system; removing the radial endobronchial ultrasound probe from the area of interest; navigating a further endobronchial tool to the area of interest; acquiring a further plurality of intraoperative images; extracting a position of the further endobronchial tool from the further plurality of intraoperative images; identifying one of the pairs in the database that corresponds most closely to the position of the further endobronchial tool; and displaying the ultrasound image corresponding to the identified one of the pairs.

In an embodiment, the further endobronchial tool is a biopsy instrument or an ablation catheter. In an embodiment, a method includes obtaining at least one preoperative image from an imaging modality; identifying, on the at least one preoperative image, at least one element located within an area of interest; obtaining at least one intraoperative image; highlighting the at least one element on the at least one intraoperative image, wherein the step of navigating the radial endobronchial ultrasound probe to the area of interest is performed using the highlighted at least one element.

In an embodiment, a method includes navigating a radial endobronchial ultrasound probe to an area of interest; selecting a confirmed position of the radial endobronchial ultrasound probe; acquiring at least one intraoperative image of the area of interest while the radial endobronchial ultrasound probe is positioned in the confirmed position; extracting a position of the radial endobronchial ultrasound probe from at least one of the at least one intraoperative image; and overlaying the confirmed position of the endobronchial ultrasound probe on at least one of the at least one intraoperative image.

In an embodiment, a method includes acquiring at least two further intraoperative images, each of the at least two further intraoperative images having a known geometric relation of the confirmed position of the radial endobronchial ultrasound probe; reconstructing the confirmed position in three-dimensional space based on the at least two further intraoperative images; and overlaying the confirmed position of the radial endobronchial ultrasound probe on at least one of the further intraoperative having a known geometric relation.

In an embodiment, a method includes removing the radial endobronchial ultrasound probe; and navigating a further endobronchial instrument to the confirmed position, whereby accurate positioning of the further endobronchial instrument is ensured. In an embodiment, the further endobronchial instrument is a biopsy instrument or an ablation catheter.

### BRIEF DESCRIPTION OF THE DRAWINGS

FIG. 1 is a flowchart of an exemplary method for using radial endobronchial ultrasound imagery to provide localization of targets.

FIG. 2 is a flowchart of an exemplary method for performing a portion of the method shown in FIG. 1.

FIG. 3A is a first image in a sample series of radial endobronchial ultrasound images that may be acquired during the performance of the exemplary method of FIG. 1.

FIG. 3B is a second image in a sample series of radial endobronchial ultrasound images that may be acquired during the performance of the exemplary method of FIG. 1.

FIG. 3C is a third image in a sample series of radial endobronchial ultrasound images that may be acquired during the performance of the exemplary method of FIG. 1.

FIG. 3D is a fourth image in a sample series of radial endobronchial ultrasound images that may be acquired during the performance of the exemplary method of FIG. 1.

FIG. 3E is a fifth image in a sample series of radial endobronchial ultrasound images that may be acquired during the performance of the exemplary method of FIG. 1.

FIG. 3F is a sixth image in a sample series of radial endobronchial ultrasound images that may be acquired during the performance of the exemplary method of FIG. 1.

FIG. 3G is a seventh image in a sample series of radial endobronchial ultrasound images that may be acquired during the performance of the exemplary method of FIG. 1.

FIG. 3H is a eighth image in a sample series of radial endobronchial ultrasound images that may be acquired during the performance of the exemplary method of FIG. 1.

FIG. 4 is a flowchart of an exemplary method for performing a portion of the method shown in FIG. 1.

FIG. 5A is an exemplary intraoperative image showing a location of a target.

FIG. 5B is an exemplary radial endobronchial ultrasound image acquired by a radial endobronchial ultrasound probe positioned as shown in FIG. 5A.

FIG. 6A is an exemplary series of radial endobronchial ultrasound images.

FIG. 6B is an exemplary three-dimensional model of a target reconstructed based on the series of radial endobronchial ultrasound images shown in FIG. 6A.

FIG. 7A is an exemplary intraoperative image showing a location of a target including a projected target profile as provided by the exemplary method of FIG. 1.

FIG. 7B is an exemplary radial endobronchial ultrasound image acquired by a radial endobronchial ultrasound probe positioned as shown in FIG. 7A.

### DETAILED DESCRIPTION

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though they may. Furthermore, the phrases “in another embodiment” and “in some other embodiments” as used herein do not necessarily refer to a different embodiment, although they may. Thus, as described below, various embodiments of the invention may be readily combined, without departing from the scope or spirit of the invention.

The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, an “absolute roll” refers to an orientation of a portion of an image with respect to an absolute (e.g., global) frame of reference. As used herein, a “relative roll” refers to the amount a current roll has changed relative to a reference roll. A reference roll is a roll which cannot be moved and may be preassigned.

In some embodiments, the method of the present invention uses imagery obtained using a radial endobronchial ultrasound (“REBUS”) probe to improve the clinical outcome of endobronchial procedures. In some embodiments, the REBUS provides radial ultrasound images inside a patient's bronchial airways. In some embodiments, the REBUS can be used in addition to the methods described in PCT/US15/56489, PCT/US14/67328, and PCT/US15/10381, which are hereby incorporated by reference in its entireties. PCT/US15/56489 discloses a method to augment an intraoperative imagery (e.g., but not limited to, X-ray, C-arm, etc.) with data from a preoperative imagery (e.g., but not limited to computerized tomography, magnetic resonance imaging) in order to assist a physician during endobronchial procedures. In some embodiments, the method includes detecting dense tissues (e.g., tissues which have a 10%, 20%, 30%, 40%, 50%, etc. increased density compared with surrounding tissues), such as lesions, inside the lungs.

In some embodiments of the methods of the present invention, an intraoperative image (e.g., an image obtained during a procedure using an imaging technique such as, but not limited to, a fluoroscopic image) and a REBUS image are acquired simultaneously. In some embodiments of the method of the present invention, an intraoperative image and a REBUS image are not acquired simultaneously (e.g., a REBUS image is acquired and a fluoroscopic intraoperative image is acquired subsequently). In some embodiments, the 3D position of the REBUS probe tip in the preoperative image is acquired using the methods, e.g., but not limited to, described in PCT/US15/56489. In some embodiments, a plurality of images of the REBUS probe tip are generated (e.g., but not limited to preoperative images). In some embodiments, the methods of the present invention further produce a database of pairs of intraoperative and REBUS images when each pair is corresponding to a specific probe tip position and orientation in the preoperative image coordinate system. In some embodiments, database can be queried or searched using the following non-limiting example: finding a nearest pair that matches the pre-marked position in the preoperative image.

In some embodiments, a method uses a set of REBUS images acquired in proximity to target area (e.g., a defined area which includes a target such as a lesion) and their position and orientation in three-dimensional space to reconstruct the outline and/or topology of the target (e.g., but not limited to, a lesion), which will be referred to herein as a “reconstructed 3D target.” In some embodiments, the reconstructed 3D target can be modified on the intraoperative image, e.g., but not limited to, projecting over or highlighting the reconstructed 3D target.

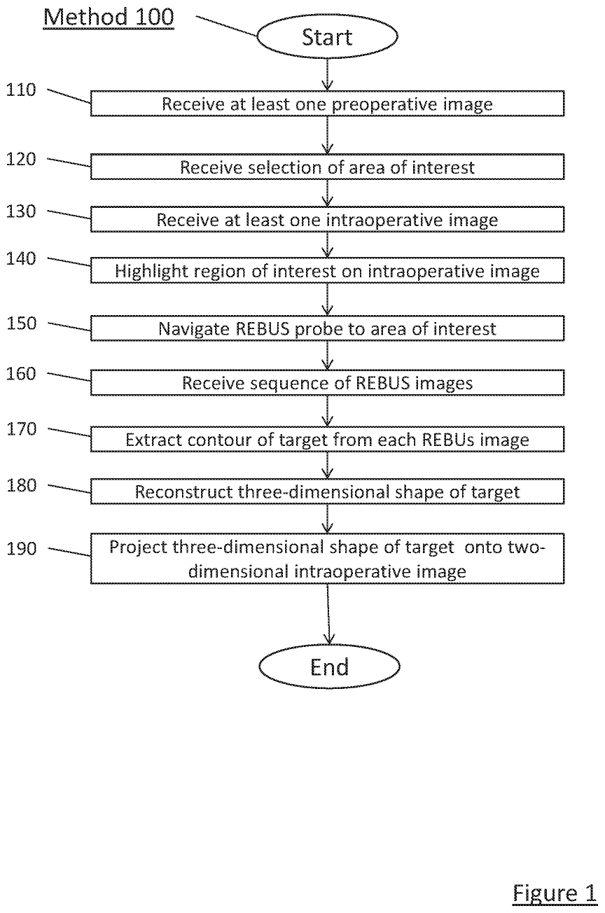

In some embodiments, a non-limiting example of target reconstruction can be performed in accordance with the method 100 shown in FIG. 1. In step 110, at least one preoperative image is acquired. In some embodiments, the preoperative image is a two-dimensional image. In some embodiments, the preoperative image is a three-dimensional image. In some embodiments, the preoperative image is any known suitable type of medical image (e.g., a computed tomography (“CT”) image). In step 120, a selection of an area of interest on the preoperative image is received. The area of interest may be, for example, a lesion.

In step 130, intraoperative images (i.e., images acquired during a procedure) are received. In some embodiments, the intraoperative images are two-dimensional images. In some embodiments, the intraoperative images are three-dimensional images. In some embodiments, the intraoperative images are any known suitable type of medical images (e.g., fluoroscope images such as X-ray images).

In step 140, a region of interest is highlighted in the intraoperative images.

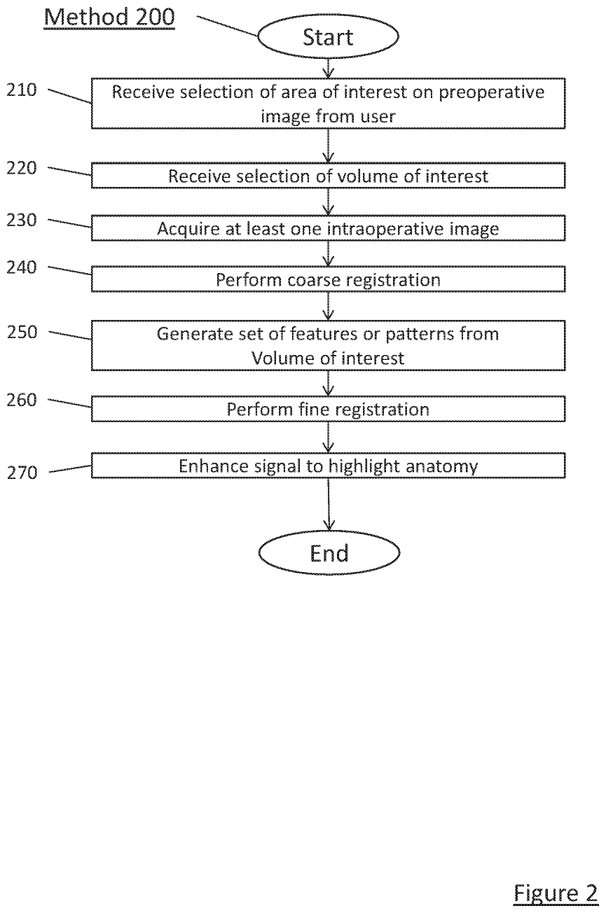

In some embodiments, the steps 100-140 are performed in accordance with the exemplary methods described in International Patent Application No. PCT/IB2015/002148, the contents of which are incorporated herein by reference in their entirety. In some embodiments, the steps 100-140 are performed in accordance with the process shown in FIG. 2.

Referring now to FIG. 2, a method 200 begins at step 210, in which a selection of an area of interest on a preoperative image, such as a CT or MRI image, is received from a user. In step 220, the volume of interest is generated the preoperative image. In some embodiments, the volume is generated in such a way that the anatomical structures in the area of interest, such as a lesion, and adjunctive anatomical structures such as bronchi or blood vessels, will be detectable on an operative image, such as fluoroscopic image. In some embodiments, a DDR image is used to evaluate detectability on fluoroscopic image. In step 230, at least one intraoperative image o is received. In an embodiment, the pose of the intraoperative modality is calculated or recorded with the at least one intraoperative image.

Continuing to refer to FIG. 2, in step 240, coarse registration between the intraoperative and preoperative images is performed, e.g., but not limited to, fluoroscopy to DDR, to evaluate a viewpoint of DDR inside a preoperative image data, such as, but not limited to, CT volume. In some embodiments, the coarse registration of step 240 is performed by applying an iterative optimization method on a viewpoint representation vector x. In some embodiments, the optimizer is initialized with initial guess x0, for example, a viewpoint corresponding to an anterior-posterior (AP) angle and positioned above the main carina. In some embodiments, for each optimization step, the following steps are performed: (1) generating a realistic DRR image; and (2) computing the similarity between the DRR image and the X-ray image. In some embodiments, coarse registration is performed as described in Kubias et al., “2D/3D Image Registration on the GPU,” University of Koblenz-Landau, Koblenz, Germany, Thomas Brunner, Siemens Medical Solutions, Forchheim, Germany, 2007, which is hereby incorporated by reference in its entirety. In some embodiments, a rib-based rigid image registration is used; for example, 2D/3D image registration, a preoperative volume (e.g. CT or MRT) is registered with an intraoperative X-ray image. In some embodiments, rigid image registration is used, where a volume can only be translated and rotated according to three coordinate axes, where a transformation is given by the parameter vector x=(tₓ, t_{y}, t_{z}, rₓ, r_{y}, r_{z}), where the parameters tₓ, t_{y}, t_{z} represent the translation in millimeters along the X, Y, and Z axes, and whereas the parameters rₓ, r_{y}, r_{z} belong to the vector r=(rₓ, r_{y}, r_{z}). In some embodiments, coarse registration is performed automatically.

In some embodiments, the coarse registration process of step 240 is performed based on an intensity-based automatic registration method using multiple intraoperative (e.g., X-ray) images and the preoperative CT volume. In some embodiments, the method is iterative. In some embodiments, for each optimization step high quality digitally reconstructed radiographs (“DRR”) are generated and then compared against acquired intraoperative (e.g., X-ray) images. In some embodiments, the method 200 uses the registration techniques disclosed in, Khamene et al., “Automatic registration of portal images and volumetric CT for patient positioning in radiation therapy,” Medical Image Analysis 10 (2006) 96-112, which is hereby incorporated by reference in its entirety. In some embodiments, such registration can be implemented, as a non-limiting example, as intensity-based and/or as feature based, depending on the specific medical application. In some embodiments, intensity-based and feature based registration are as described by David et al., “Intensity-based Registration versus Feature-based Registration for Neurointerventions,” Medical Vision Laboratory, Dep't of Engineering Science, University of Oxford, England, which is hereby incorporated by reference in its entirety. In some embodiments, point-based registration is implemented using known anatomical landmarks on a patient's chest. In some embodiments, at least one known landmark can be marked on a CT image and/or fluoroscopic image. In some embodiments, special markers are attached to the patient's chest during procedure to improve/increase detectability on a fluoroscopic image.

Continuing to refer to FIG. 2, in step 250, a set of features or patterns, depending on the desired registration method, is generated from a volume of interest of the preoperative image. In some embodiments, when the soft tissue structures of a patient are observed and move relative to the ribs of the patient, the viewpoint calculated during coarse registration at 240 is approximated within the known tolerance. In some embodiments, the set of patterns generated in step 250 allow performing the fine-tuning (i.e., fine registration) of the viewed area in the following step. In step 260, fine registration is implemented to find the best fit between each of the features or patterns, depending on the registration method, generated at 250 and area of interest on intraoperative image. In some embodiments, fine registration includes intensity-based fine registration (i.e., template matching), where the approach is initiated with an intensity-based pattern from a pre-operative or a reference imaging modality. In some embodiments, the signal from an intraoperative image contains noise and scale and is measured within the area of interest. In some embodiments, the fine registration process of step 260 is applied for each intraoperative image and includes the following steps: (1) comparing the intensity-based pattern from a pre-operative or a reference imaging modality to an intraoperative image and finding the position in intraoperative image with maximal similarity to the pattern; (2) calculating the two-dimensional shift between the new and previous position of the pattern; and (3) correcting the coarse registration using the calculated two-dimensional shift In some embodiments, fine registration is performed as described in Mahalakshmi et al., “An Overview of Template Matching Technique in Image Processing,” School of Computing, SASTRA University, Thanjavur, Tamil Nadu, India, Research Journal of Applied Sciences, Engineering and Technology 4(24): 5469-5473, 2012, which is hereby incorporated by reference in its entirety.

In some embodiments, the fine registration process of step 260 includes the steps of: (1) Feature Identification: identifying a set of relevant features in the two images, such as edges, intersections of lines, region contours, regions, etc; (2) Feature Matching: establishing correspondence between the features (i.e., each feature in the sensed image is be matched to its corresponding feature in the reference image); each feature is identified with a pixel location in the image, and these corresponding points are usually referred to as control points; (3) Spatial Transformation: determining the mapping functions that can match the rest of the points in the image using information about the control points obtained in the previous step; and (4) Interpolation: resampling the sensed image using the above mapping functions to bring it into alignment with the reference image. Some embodiments use an area-based approach, which is also referred to as correlation-like methods or fine registration (i.e., template matching), such as described in Fonseca et al., “Registration techniques for multisensor remotely sensed imagery,” PE & RS-Photogrammetric Engineering & Remote Sensing 62 (9), 1049-1056 (1996), which describes the combination of feature detection and feature matching. In some embodiments, the method 200 is suited for templates which have no strong features corresponding to an image, since the templates operate directly on the bulk of values. In some embodiments, matches are estimated based on the intensity values of both image and template. In some embodiments, techniques that are used include squared differences in fixed intensities, correction-based methods, optimization methods, mutual information, or any combination thereof. In some embodiments, fine registration is performed automatically. In some embodiments, fine registration includes aligning a 2D projection of an anatomical structure from a CT scan obtained through coarse registration with correspondent anatomical structure extracted from fluoroscopic image.

Continuing to refer to FIG. 2, in step 270, the matched signal from the fine registration step is enhanced to highlight the anatomy found in the area of interest as shown in the preoperative image. In some embodiments, in addition to highlighting the signal from intraoperative image, the signal sourcing from the reference image can be overlaid on the display/image. In some embodiments, the combination of the original signal from the intraoperative image, the simulated signal from the reference image, and any planning information can be displayed according to application configuration or upon the user request.

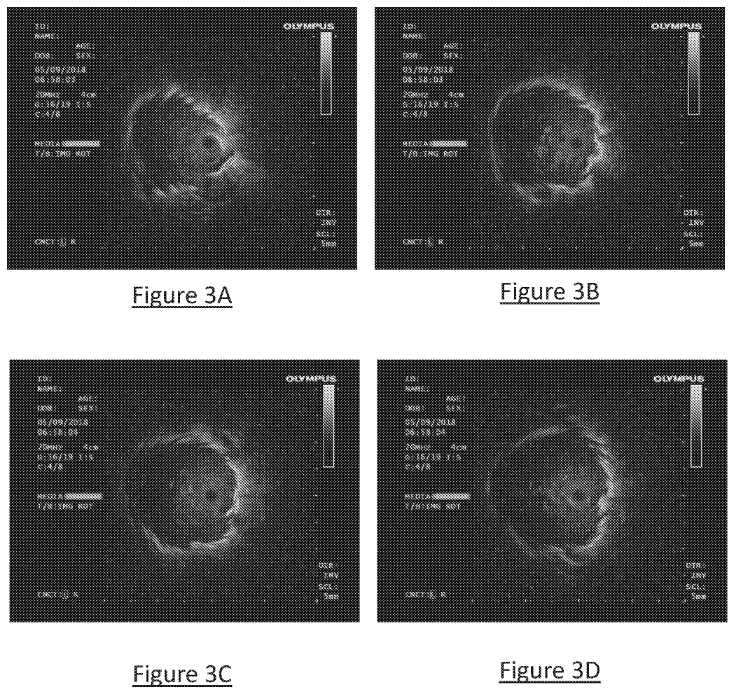

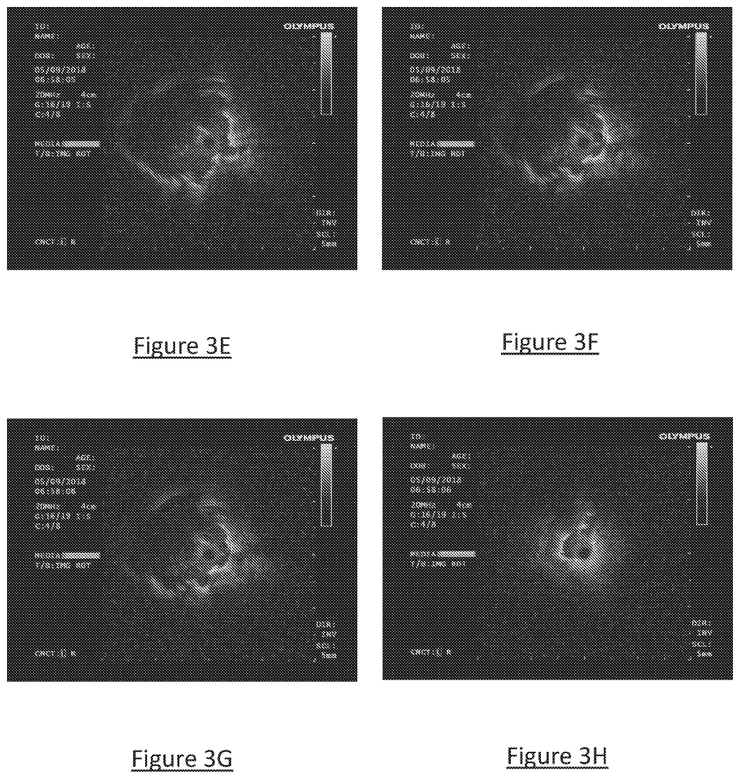

Referring back to FIG. 1, in step 150, a REBUS probe is navigated to an area of interest near the target. In some embodiments, navigation is accomplished through the use of enhanced imagery as generated by the steps described above. In step 160, a sequence of REBUS images is acquired as the REBUS probe is moved along the bronchial passageway. As noted above, in some embodiments, each such REBUS image represents a cross-sectional “slice” of the patient's tissue. FIGS. 3A-3H show a representative set of REBUS images. In step 170, for each REBUS image from the sequence, the target contour is extracted from the ultrasound image relatively to the REBUS probe tip which is detected both on REBUS image and in the intraoperative image. In some embodiments, a target contour is visible on the radial endobronchial ultrasound images as a curve having a strong intensity gradient. In some embodiments, such curves are detected by calculating an image gradient, applying a threshold, and calculating connected components that comprise the detected curve pixels. In some embodiments, one of the methods described in Noble et al., “Ultrasound Image Segmentation: A Survey”, IEEE TRANSACTIONS ON MEDICAL IMAGING, VOL. 25, NO. 8, (AUGUST 2006) is used. In some embodiments, a machine learning approach is used based on training a neural network to detect such contours in a more robust fashion. In some embodiments, the training process uses a large number of annotated sample images. In some embodiments, a target contour is detected from the set of REBUS slices in accordance with the techniques described in Shen et al., “DeepContour: A Deep Convolutional Feature Learned by Positive-sharing Loss for Contour Detection”, CVPR 2015.

In step 180, the three-dimensional shape of the target is reconstructed based on the extracted target contours from the REBUS images and the known position and orientation of the probe tip from the intraoperative image. In some embodiments, the extracted target contours and the known position and orientation of the probe tip define a REBUS-based real space. In some embodiments, the process of reconstruction includes the steps of: (1) mapping each two-dimensional target contour point to a voxel in the REBUS-based real space; (2) traversing the REBUS-based real space and generating a three-dimensional target model by marking every voxel surrounded by or belonging to contour points as an target voxel; and (3) applying a surface-extraction algorithm to generate a three-dimensional target surface from the reconstructed target model. In some embodiments, the 3D volume is reconstructed from the set of REBUS slices in accordance with the techniques described in Zang et al., “3D Segmentation and Reconstruction of Endobronchial Ultrasound”, Medical Imaging 2013: Ultrasonic Imaging, Tomography and Therapy (Vol. 8675).

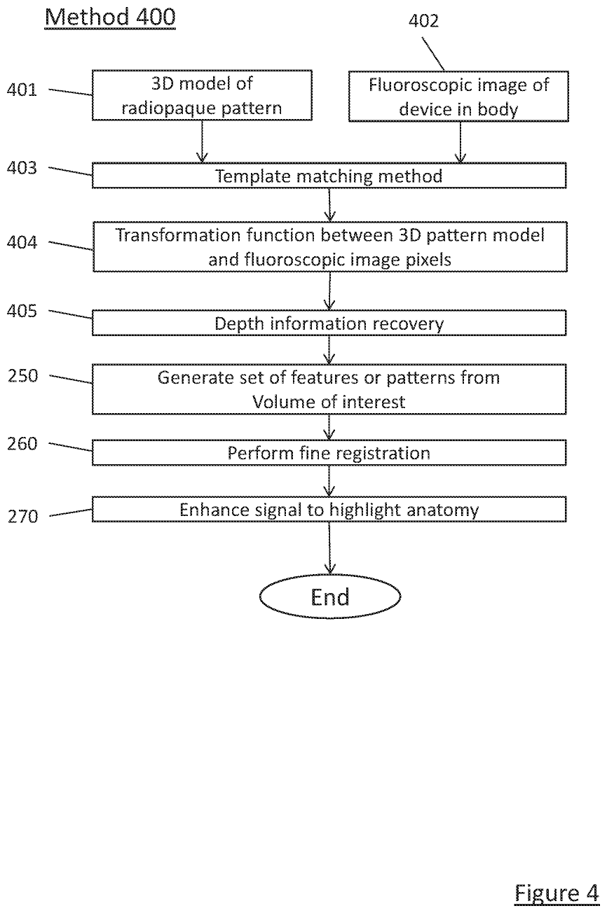

In some embodiments, the position and orientation of the REBUS probe may be identified using the techniques described in International Pat. App. No. PCT/IB17/01376, the contents of which are incorporated herein by reference in their entirety. In some embodiments, a radiopaque pattern is provided on the REBUS probe in order to facilitate such identification. In some embodiments, the position and orientation of the REBUS probe may be performed in accordance with the process shown in FIG. 4.

FIG. 4 shows an exemplary method 400 for determining the position and orientation of a REBUS probe within a patient's body. The method 400 receives, as input, a density model (401) of the radio opaque material along the device (e.g., a pattern) and fluoroscopic image data (402) showing the device positioned within the patient's body. In some embodiments, a transformation function (404) between the model and the image pixels is calculated using a template matching method (403). In some embodiments, the template matching method is performed as follows: when a portion of a pattern of radio opaque material is visible, a one-dimensional translation (e.g., correlation) between the imaged pattern and the density function can be calculated. The relation between the radio opacity of the device and the gray-scale levels can be used for this purpose. In some embodiments, a template matching method that searches for the highest correlation between the gray-scale levels of the visible segment of the device in the image and the radio opaque density profile of the device is used. Such a method is robust to occlusion and noise caused by objects that are behind or above the device with respect to the projection direction from an X-ray tube to an image intensifier. In some embodiments, a correlation function between the device's partial image and the device's pattern of radio opaque material density is calculated. In some embodiments, the transformation function is used for depth information recovery (405).

In step 190, the reconstructed target shape is projected. In some embodiments, the complete or partial 3D target is be segmented from this volume and projected over the intraoperative image combining the values of the projected and source volume depending on the application need. In some embodiments, the 3D target is highlighted over the inter-operative image. In some embodiments, the full or partial target is used for the registration between the preoperative image and postoperative image.

In some embodiments, a 3D target reconstructed from REBUS images is registered to a 3D volume sourced from the preoperative image. In an embodiment, the registration between the two volumes is based on matching voxel intensity values. In an embodiment, the registration between the 3D target and the volume is performed by generating a binary volume from the 3D target shape and then registering two volumes based on matching voxel intensity values. In an embodiment, the registration between the 3D target and the volume is performed by extracting geometric shapes of anatomical structures from the volume and then matching these geometric shapes with the shape of the 3D target. In an embodiment, the registration between the 3D target and the volume is based on co-aligning the centers of mass or moments of inertia. In some embodiments, the pose of an intraoperative image relative to a preoperative image can be further calculated from the known position (e.g., location and orientation) of the REBUS probe on the intraoperative image.

In some embodiments, a 3D target reconstructed from REBUS images is registered to the 3D volume reconstructed from plurality of intraoperative images, wherein the reconstruction methods, not limited to, described in PCT/US15/56489.

In some embodiments, a 3D target reconstructed from REBUS images is used as a prior for volume reconstruction from plurality of intraoperative images. In some embodiments, the area of interest can be segmented on reconstructed 3D target. In some embodiments, only a partial shape or area of interest is reconstructed from REBUS images, registered to the volume sourcing from the preoperative image and enhanced or accomplished using the information from preoperative image to obtain additional information in the volume reconstructed from intraoperative images.

In some embodiments, the 3D target is reconstructed from different interoperative images, such as a set of fluoroscopic images or CT images, with known relative pose. In some embodiments, such reconstruction can be performed using a back projection algorithm or any other reconstruction algorithm utilized in computational tomography. In some embodiments, a 3D target or area of interest reconstructed from REBUS images can be co-registered with compatible 3D volume or area of interest sourcing additional interoperative image, preoperative image or combination of thereof.

In some embodiments, the non-limiting examples above rely on determination of the REBUS probe tip position and orientation with respect to the intraoperative image. In some embodiments, the methods disclosed in International Pat. App. No. PCT/IB17/01376 can be used in the methods of the present invention. In some embodiments, a radio opaque pattern attached to the catheter can be used to determine its pose with respect to the intraoperative imaging device. In some embodiments, a helical spring pattern, which is asymmetric relatively to the axes of catheter rotation, can be used to determine the relative or absolute roll for each REBUS slice. In some embodiments, an angular measurement device attached to the probe tip of the catheter can be used to determine the relative or absolute roll for each REBUS slice. In some embodiments, adjacent frames can be stabilized by minimizing rotation difference between these frames.

In some embodiments, a REBUS image with an associated position and orientation is used to locate bronchial tree bifurcations on the intraoperative image. In some embodiments, such bifurcations may be used as additional fiducial points in the registration process described in International Pat. App. No. PCT/US15/56489. In some embodiments, an airway bifurcation may be visible on a REBUS image and can be detected by means of image processing (e.g., but not limited to, highlighting). In some embodiments, since (1) at least one image of REBUS is of a probe tip acquired by an intraoperative device, and at the same time (2) at least one image of bifurcation is acquired by REBUS itself, the position and orientation of the bifurcation may be marked in the intraoperative image. In some embodiments, the bifurcation position in the intraoperative image and its corresponding position in the preoperative image as additional fiducial points to improve to accuracy for the registration process can obtained using the methods described in, e.g., but not limited to, PCT/US15/56489.

In some embodiments, a location of the REBUS probe at the selected position of a REBUS image can be marked or confirmed on a corresponding intraoperative image; this may be referred to as “REBUS confirmation”. In some embodiments, the location of the tip of the REBUS probe as seen in the intraoperative image can be marked, stored and shown at any time, even after the REBUS probe has been retracted and another device (e.g., other endobronchial instruments or endotherapeutic accessories) has been introduced into the same area inside the patient. In some embodiments, a compensation for respiratory motion and tissue deformation caused by endobronchial instruments may be provided, whereby the displayed position of selected and stored location of the REBUS are adjusted. In an embodiment, the movement compensation is performed by tracking anatomical structures and/or instruments. In some embodiments, the position of the REBUS probe corresponding to the “REBUS confirmation” can be extracted from multiple intraoperative images having different poses with known geometric relation, thereby providing the 3D reconstruction of the REBUS probe and, particularly, the calculated position of the tip of the REBUS probe. In some embodiments, the 3D location of the “REBUS confirmation” location can be projected onto any future intraoperative image(s).

### EXAMPLES

The clinical applications of the exemplary embodiments can be illustrated through the following prophetic examples:

1) The exemplary embodiments may provide the physician with REBUS images during the time when the REBUS probe is retracted and replaced by other endobronchial tool, such as biopsy forceps or ablation probe.

2) Where the target is a lesion, by reconstructing the shape of the lesion in three dimensions with respect to the position of the REBUS probe, the maximal distance from the center of the probe to the boundary of the lesion can be determined. This information may provide an appropriate margin size for an ablation procedure when the REBUS probe has been removed and replaced by an ablation catheter. For example, the margin can be calculated by multiplying a constant by the maximal distance from the center of the REBUS probe to the boundary of the lesion.

3) The desired area of interest can be marked on the acquired REBUS images and an endobronchial tool can be guided to the desired preplanned location (e.g., marking on the acquired REBUS images).

4) A method to obtain an accurate localization of the target during, e.g., but not limited to, an endobronchial biopsy or ablation. The method can be performed using the following steps:

1. Endobronchial navigation to the target region. The navigation may be performed using, e.g., but not limited to, the navigation method described in PCT/US15/56489.

2. Acquire REBUS images in the target area.

3. Extract the target from the REBUS images and project it on the intraoperative image.

4. Position the endobronchial tool in the target center and perform an ablation or biopsy.

5. If ablation was performed and the anatomy changed, steps 2-4 may be performed again.

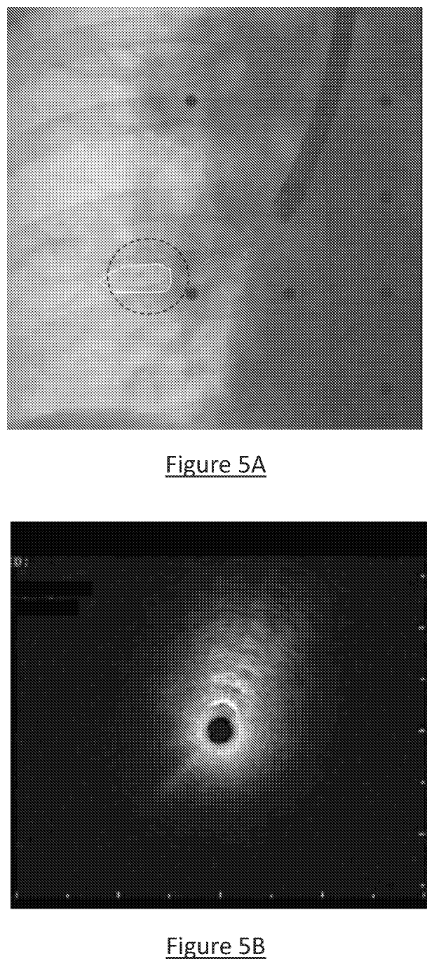

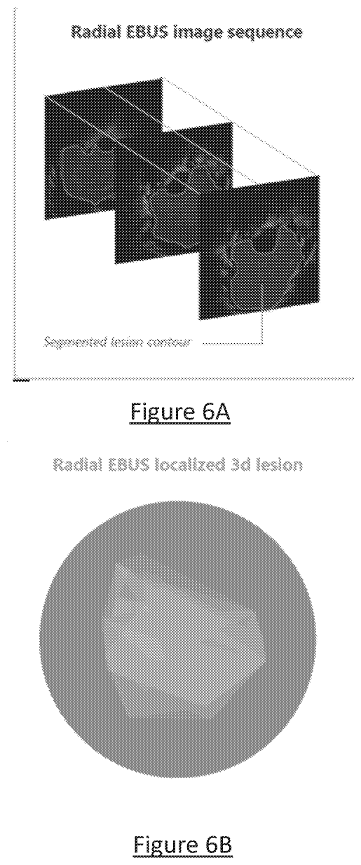

FIGS. 5A and 5B show an embodiment of a target area radial EBUS scan generated using the methods of some embodiments of the present invention. The dotted circle defines the target area in FIG. 5A. The white outlined portion in FIG. 5A is the target defined using the method of the present invention.

FIG. 6A shows a sequence of radial EBUS images of a lesion that may be obtained during the course of the exemplary method shown in FIG. 1. FIG. 6B shows a three-dimensional contour of the lesion that may be reconstructed using three-dimensional calculations based on the sequence of radial EBUS images shown in FIG. 6A by the exemplary method of FIG. 1.

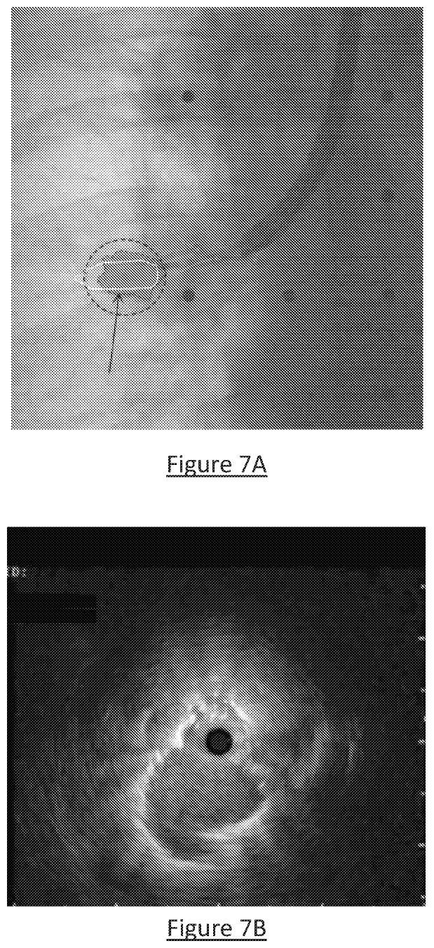

FIGS. 7A and 7B show images generated using an embodiment of the methods of the present invention, and show a real time, localized view with augmentation using radial EBUS. The dotted circle defines the target area in FIG. 7A. The white outlined portion in FIG. 7A is the target defined using the methods described herein. The arrow points to the region which has been determined to be the target using the methods described herein.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

What is claimed is:

1. A method, comprising:
obtaining at least one preoperative image from an imaging modality;
identifying, on the at least one preoperative image, at least one element located within an area of interest;
obtaining at least one intraoperative image;
highlighting the at least one element on the at least one intraoperative image;
navigating a radial endobronchial ultrasound probe to the area of interest using the at least highlighted at least one element;
acquiring a plurality of radial endobronchial ultrasound images;
extracting a plurality of two-dimensional representations of the element, each of the plurality of two-dimensional representations of the element being extracted from a corresponding one of the plurality of radial endobronchial ultrasound images;
reconstructing a three-dimensional representation of the element from the plurality of two-dimensional representations of the element; and
projecting a two-dimensional projection of the three-dimensional representation of the element on at least one of the at least one intraoperative image.

2. The method of claim 1, wherein the step of projecting the two-dimensional projection of the three-dimensional representation of the element on the at least one of the at least one intraoperative image is performed in real time.

3. The method of claim 1, further comprising:
removing the radial endobronchial ultrasound probe from the area of interest; and
navigating a further endobronchial tool to the area of interest.

4. The method of claim 3, further comprising:
performing a procedure on the element using the further endobronchial tool.

5. The method of claim 4, further comprising:
removing the further endobronchial tool;
navigating the radial endobronchial ultrasound probe to the area of interest;
acquiring a plurality of updated radial endobronchial ultrasound images;
extracting a plurality of updated two-dimensional representations of the element, each of the plurality of updated two-dimensional representations of the element being extracted from a corresponding one of the plurality of updated radial endobronchial ultrasound images; and
reconstructing an updated three-dimensional representation of the element from the plurality of two-dimensional representations of the element.

6. The method of claim 1, further comprising:
calculating distances between a center of the radial endobronchial ultrasound probe and a plurality of boundary points on a boundary of the target; and
estimating a margin size for an ablation based on a maximum one of the distances.

7. The method of claim 1, wherein the at least one intraoperative image includes an X-ray.

8. The method of claim 1, wherein the three-dimensional representation of the element is used as a prior for volume reconstruction from at least one of the intraoperative images.

9. The method of claim 1, further comprising:
registering the three-dimensional representation of the target to a three-dimensional computed tomography volume; and
projecting the three-dimensional representation of the element from the three-dimensional computed tomography volume on at least one of the at least one intraoperative image.

10. The method of claim 9, wherein the three-dimensional computed tomography volume is a preoperative computed tomography scan volume or a three-dimensional computed tomography volume reconstructed from the at least one intraoperative image.

11. A method, comprising:
navigating a radial endobronchial ultrasound probe to an area of interest;
acquiring a plurality of radial endobronchial ultrasound images and a plurality of intraoperative images, each of the plurality of radial endobronchial ultrasound images corresponding to one of the plurality of intraoperative images and to a different position of the ultrasound probe;
extracting a radial endobronchial ultrasound probe tip position from each of the intraoperative images;
generating a database of pairs of the intraoperative and endobronchial ultrasound images, each pair corresponding to a specific probe tip position and orientation in the preoperative image coordinate system;
removing the radial endobronchial ultrasound probe from the area of interest;
navigating a further endobronchial tool to the area of interest;
acquiring a further plurality of intraoperative images;
extracting a position of the further endobronchial tool from the further plurality of intraoperative images;
identifying one of the pairs in the database that corresponds most closely to the position of the further endobronchial tool; and
displaying the ultrasound image corresponding to the identified one of the pairs.

12. The method of claim 11, wherein the further endobronchial tool is a biopsy instrument or an ablation catheter.

13. The method of claim 11, further comprising:
obtaining at least one preoperative image from an imaging modality;
identifying, on the at least one preoperative image, at least one element located within an area of interest;
obtaining at least one intraoperative image;
highlighting the at least one element on the at least one intraoperative image,
wherein the step of navigating the radial endobronchial ultrasound probe to the area of interest is performed using the highlighted at least one element.

14. A method, comprising:
navigating a radial endobronchial ultrasound probe to an area of interest;
selecting a confirmed position of the radial endobronchial ultrasound probe;
acquiring at least one intraoperative image of the area of interest while the radial endobronchial ultrasound probe is positioned in the confirmed position;
extracting a position of the radial endobronchial ultrasound probe from at least one of the at least one intraoperative image; and
overlaying the confirmed position of the endobronchial ultrasound probe on at least one of the at least one intraoperative image.

15. The method of claim 14, further comprising:
acquiring at least two further intraoperative images, each of the at least two further intraoperative images having a known geometric relation of the confirmed position of the radial endobronchial ultrasound probe;
reconstructing the confirmed position in three-dimensional space based on the at least two further intraoperative images; and
overlaying the confirmed position of the radial endobronchial ultrasound probe on at least one of the further intraoperative having a known geometric relation.

16. The method of claim 14, further comprising:
removing the radial endobronchial ultrasound probe; and
navigating a further endobronchial instrument to the confirmed position, whereby accurate positioning of the further endobronchial instrument is ensured.

17. The method of claim 16, wherein the further endobronchial instrument is a biopsy instrument or an ablation catheter.
